(19) United States
(12) Patent Application Publication
(10) Pub. No.: US 2013/0253455 A1
(43) Pub. Date: Sep. 26, 2013

(54) EXPANDABLE OSTOMY APPLIANCE

(71) Applicant: HOLLISTER INCORPORATED, Libertyville, IL (US)

(72) Inventors: Brock E. Masters, Des Plaines, IL (US); Kimberly R. Hansford, Winthrop Harbor, IL (US); Tze Wan Pansy Chung, Fox River Grove, IL (US)

(73) Assignee: HOLLISTER INCORPORATED, Libertyville, IL (US)

(21) Appl. No.: 13/803,917

(22) Filed: Mar. 14, 2013

Related U.S. Application Data
(60) Provisional application No. 61/614,258, filed on Mar. 22, 2012.

(51) Int. Cl. A61F 5/445

(52) U.S. Cl. CPC A61F 5/445
USPC 604/332

(57) ABSTRACT

An expandable ostomy pouch and an expandable ostomy cap include a first portion and a second portion sharing a common cavity. The first portion and the second portion are configured such that the second portion can be inverted and inserted within the first portion in a compacted state. The second portion can be manually pulled out or can be expanded due to gravity and/or due to elevated pressure within the pouch 10 due to the stomal discharge, as the cavity is filled with stomal discharge.

### CROSS-REFERENCE TO RELATED APPLICATION DATA

This application claims the benefit of priority of Provisional U.S. Patent Application Ser. No. 61/614,258, filed Mar. 22, 201:2 entitled, “EXPANDABLE OSTOMY APPLIANCE.”

### BACKGROUND

The present disclosure relates to ostomy appliances, and more particularly to stoma caps and ostomy pouches having expandable waste collection capacities.

Ostomy appliances for collecting body waste, such as ostomy pouches, are used by patients who have had surgery such as a colostomy, ileostomy, or urostomy. Ostomy pouches typically include flat, opposing side walls secured together along their edges to define a collection cavity. One of the side walls is provided with an opening to receive a stoma, and means to secure the pouch to the user, such as an adhesive barrier, so that body waste discharged through the stoma is received within the cavity. At its lower end, the ostomy pouch may have a discharge opening which may be closed during collection of the body waste material but may be opened for draining the body waste material from the pouch after a period of use. Alternatively, the ostomy pouch may be designed for a single use, in which case it will not be provided with a discharge opening since the entire pouch will be discarded after it has substantially tilled with stomal discharge.

Ostomy pouches are available in various sizes for different collecting capacity needs. For example, a user will want to use a large ostomy bag over night, but may desire to use a smaller ostomy pouch for discretion during public activities or when exercising. Occasionally, for example, during exercising, intimacy, or showering, the user may prefer using a smaller device, such as a stoma cap, which is designed to be just large enough to cover the stoma for increased discretion and mobility.

Ostomy caps and small ostomy pouches are preferred by users for obvious reasons. However, as stomal discharge cannot be regulated at will, stoma caps and small ostomy pouches often cannot provide sufficient collection capacity to contain a sudden outflow of stomal discharge, resulting in leakage and embarrassment to users.

Accordingly, there is a need for an improved stoma cap and ostomy pouch that is small enough to provide the discretion desired by the user, yet can also provide a sufficient collection capacity to contain sudden outflows of stomal discharge.

### BRIEF SUMMARY

An expandable stoma cap and an expandable ostomy pouch are provided according to various embodiments. The expandable stoma cap and the expandable ostomy pouch are small in site in a compacted state to provide a higher degree of discretion for users, and configured to expand to provide an increased waste collection capacity to accommodate sudden outflows of stomal discharge.

In one aspect, an expandable ostomy pouch is provided. The expandable ostomy pouch includes a first portion and a second portion. The first portion and the second portion may be defined by a bodyside wall and an outer wall, which may be sealed along their peripheral edges to define a cavity. The cavity may be continuous through the first portion and the second portion. The expandable ostomy pouch also includes an inlet opening arranged on the bodyside wall of the first portion for receiving a stoma.

The second portion may be configured to be inverted and inserted within the first portion, such that the second portion is substantially completely hidden and contained within the first portion in a compacted state, and may be substantially completely released and exposed from the first portion in an expanded state.

The expandable ostomy pouch may be configured to expand from the compacted state to the expanded state by manually pulling the second portion from the first portion, or due to gravity and/or due to elevated pressure within the pouch due to the introduction of stomal discharge into the pouch, as the cavity is filled with stomal discharge. Further, the first portion and the second portion may be configured such that the second portion is fitted securely within the first portion and the second portion does not release from the first portion during exercise or other user movement without being manually pulled out or expanded by stomal discharge.

In one embodiment, the expandable ostomy pouch has a peanut-like shape, in which the first portion has a substantially circular shape and the second portion has a substantially circular shape. In such an embodiment, the second portion may be sized to be slightly smaller than the first portion, such that the second portion can be inverted and securely fitted within the first portion.

In another embodiment, the first portion has a substantially square shape and the second portion has a substantially rectangular shape, in which a width of the second portion is smaller than a width of the first portion, and/or a length of the second portion is smaller than a length of the first portion, such that the pouch has substantially the same shape and size as the first portion when in a compacted state with the second portion contained within the first portion.

The expandable ostomy pouch can be configured as a drainable pouch including an outlet opening defined at an end of the second portion and a closing means for closing the outlet opening. In this configuration, the outlet opening is closed prior to being inverted and inserted within the first portion in the compacted state. Alternatively, the expandable ostomy pouch can be a “closed” pouch without an outlet opening.

In some embodiments, the waste collection capacity of the expandable ostomy pouch in the expanded state is at least 50% greater than that in the compacted state, and preferably at least 100% greater than that in the compacted state.

The expandable ostomy pouch can also include a non-woven material arranged on an outer surface of at least one of the bodyside wall and the outer wall, in which the non-woven material extends along the first portion only.

In one embodiment, the expandable ostomy pouch further includes an adhesive skin barrier arranged about the inlet opening for attaching the expandable ostomy pouch to a user. Alternatively, the expandable ostomy pouch includes a coupling arranged about the inlet opening for attaching the expandable ostomy pouch to a mating bodyside coupling of an adhesive barrier attached on a user.

Further, the second portion of the expandable ostomy pouch can include a tab for facilitating expansion of the pouch from the compacted state to the expanded state. The tab is configured such that a user can grasp the tab and pull out the second portion from within the first portion. In one embodiment, the tab is defined by a heat sealed portion in a lower part of the second portion.

In some embodiments, the second portion in the compacted state is attached to the first portion on respective inner surface of the outer wall with a water-soluble adhesive material.

In another embodiment, the expandable ostomy pouch includes a filter arranged on an inner surface of the outer wall of the first portion. In such an embodiment, the inverted second portion is arranged between the inlet opening and the filter in the compacted suite to at least partially shield the filter from liquid and solid stomal discharge to extend filter life and enhance filter performance that may otherwise be degraded h partial or total blockage of the filter by stomal discharge.

In another aspect, an expandable ostomy cap is provided. The expandable ostomy cap includes a cap portion, which is sized large enough to cover a stoma and to support a means for attaching the expandable ostomy cap to a user. The expandable ostomy cap also includes a tail portion. The cap portion and the tail portion may be defined by a bodyside wall and an outer wall, which may he sealed along their peripheral edges to define a cavity. The cavity is continuous through the cap portion and the tail portion. The expandable ostomy cap also includes an inlet opening arranged on the bodyside wall of the cap portion for receiving a stoma. The tail portion may be configured to be inverted and inserted within the cap portion in a compacted state such that the tail portion is substantially completely contained and hidden within the cap portion, and the second portion may be substantially completely released and exposed from the first portion in an expanded state.

In one embodiment, the cap portion has a substantially square shape, in which each side of the cap portion is between about 3.5 inches and about 5 inches. In another embodiment, the cap portion has a substantially circular shape with a diameter between about 3.5 inches and about 5 inches.

The cap portion and the tail portion may be configured such that the tail portion is fitted securely within the cap portion and does not expand out of the cap portion during exercise or other user movement without being manually pulled out or expanded by stomal discharge.

In one embodiment, a waste collection capacity of the expandable ostomy cap in the expanded state is at least 100% greater than that in the compacted state.

In some embodiments, the tail portion is attached to the cap portion in the compacted state on an inner surface of the outer wall of the cap portion with a water-soluble material.

Other aspects, objectives and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a schematic view of an expandable ostomy pouch in an expanded state according to an embodiment;

FIG. 2 is a schematic cross-sectional view of the expandable ostomy pouch of FIG. 1 taken along line A-A;

FIG. 3 is a schematic view of the expandable ostomy pouch of FIG. 1 in a compacted state;

FIG. 4 is a schematic cross-sectional view of the expandable ostomy pouch of FIG. 3 taken along line B-B;

FIG. 5 is a schematic view of fan expandable ostomy cap in an expanded state according to an embodiment;

FIG. 6 is a schematic cross-sectional view of the expandable ostomy cap of FIG. 5 taken along line

FIG. 7 is a schematic view of the expandable ostomy cap of FIG. 5 in a compacted state;

FIG. 8 is a schematic cross-sectional view of the expandable ostomy cap of FIG. 7 taken along line D-D:

FIG. 9 is a schematic cross-sectional view of an alternate embodiment of the expandable cap in a compacted state;

FIG. 10A is a schematic view of an expandable ostomy pouch including a discharge outlet in an expanded state according to another embodiment;

FIG. 10B is a schematic view of the expandable ostomy pouch of FIG. 10A with the discharge outlet closed; and

FIG. 11 is a schematic view of an ostomy pouch in an expanded stated according an embodiment.

### DETAILED DESCRIPTION

While the present disclosure is susceptible of embodiment in various forms, there is shown in the drawings and will hereinafter be described a presently preferred embodiment with the understanding that the present disclosure is to be considered an exemplification and is not intended to limit the disclosure to the specific embodiment illustrated. The words “a” or “an” are to be taken to include both the singular and the plural. Conversely, any reference to plural items shall, where appropriate, include the singular.

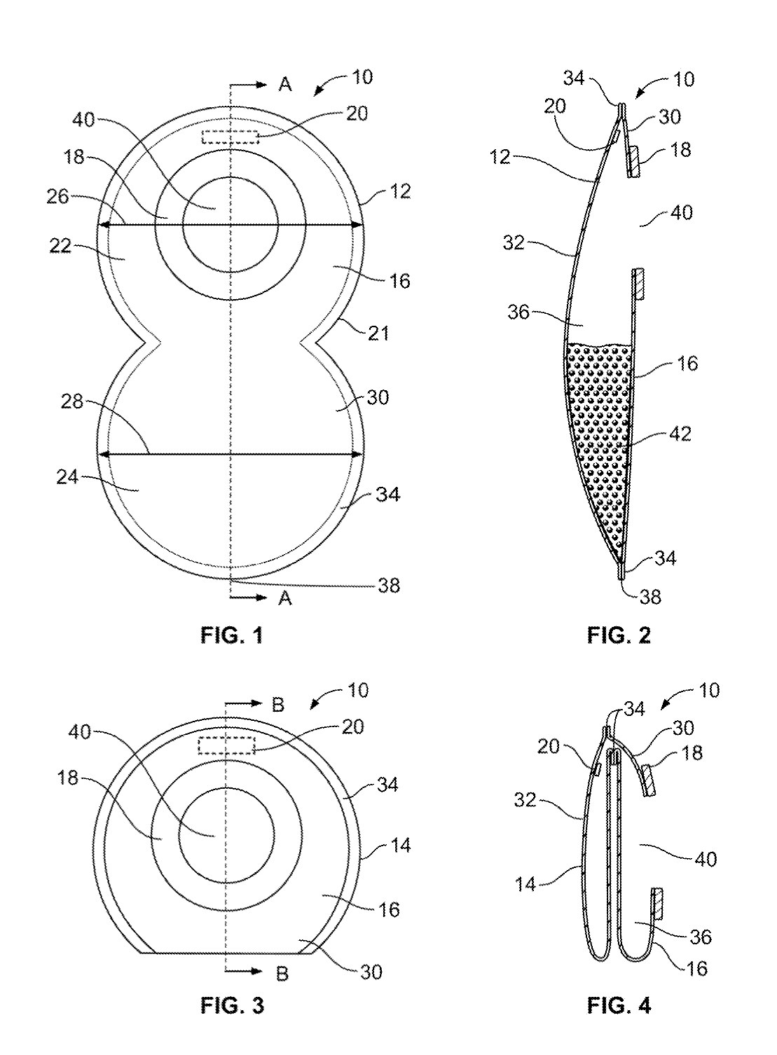

Referring now to the figures, FIGS. 1-4 show an embodiment of an expandable ostomy pouch 10. FIGS. 1 and 2 illustrate the expandable pouch 10 in an expanded state 12, and FIGS. 3 and 4 illustrate the expandable ostomy pouch 10 in a compacted state 14. The expandable ostomy pouch 10 includes, generally, a pouch 16, a skin barrier 18, and a filter 20. The pouch 16 can be provided in various different sizes.

As shown in FIG. 1, the expandable ostomy pouch 10 may have a “peanut” shape body 21, and includes an upper rounded portion 22 and a lower rounded portion 24. In a preferred embodiment, the upper and lower portions are circular or near-circular. The upper rounded portion 22 has a diameter 26, which is slightly greater than a diameter 28 of the lower rounded portion 24, such that the lower rounded portion 24 can be inverted and fit in the upper rounded portion 22 to form the expandable pouch 10 in the compacted state 14. The upper portion 22 and the lower portion 24 may be configured such that, in the compacted state 14, the lower portion 24 remains inverted and fitted in the upper portion 22 until a user manually deploys the lower portion 24 by a pulling motion, or the pouch 16 is expanded due to gravity as the pouch 16 is filled with stomal discharge 42.

The pouch 16 may include a bodyside wall 30 and an outer wall 32, which may be sealed together along their outer peripheral edges 34 to define a cavity 36 for collecting stomal discharge 42. Each the bodyside wall 30 and the outer wall 32 may be formed of the same material or different materials. For example, each of the walls 30, 32 can be formed of a multi layer film, which may be clear or opaque.

In some embodiments, a non-woven material may be provided on the outer surface of the bodyside wall 30 and/or the outer wall 32.

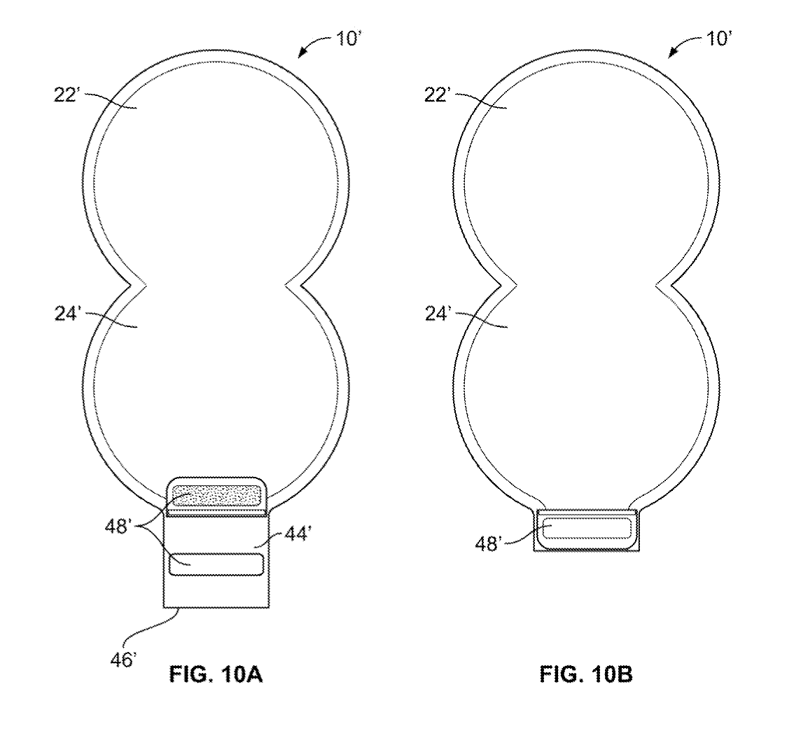

The expandable ostomy pouch 10 shown in FIGS. 1-4 has a closed end 38 without a discharge outlet. In other embodiments, the pouch may include a discharge outlet which may be closed during collection of the stomal discharge and opened for periodic draining of the pouch. For example, the pouch may include an outlet that may be secured in a closed configuration by a separate clamping device, or alternatively, the pouch may include an integrated closure mechanism, such as, for example, one of the closure mechanisms disclosed in Villefrance, et al., U.S. Pat. No. 7,879,015, and Mandzij, et al., U.S. Pat. No. 7,879,016, both of which are commonly assigned with the present application and are incorporated herein by reference. FIGS. 10A and 10B illustrate an expandable ostomy pouch 10′ including a neck portion 44′ with a discharge outlet 46′. The neck portion 44′ is provided with a closure mechanism 48′. In such embodiments, the neck portion 46′ is rolled up and securely closed as shown in FIG. 10B before a lower rounded portion 24′ is inverted and fitted within an upper rounded portion 22′ to form the pouch in a compacted state.

The bodyside wall 30 may be provided with an inlet opening 40 to receive a stoma. The adhesive skin barrier 18 is arranged about the inlet opening 40 for attachment around the stoma. The expandable pouch 10 may also be provided with filter 20, such as those disclosed in Nolan, U.S. Pat. No. 3,952,727, and Botten, U.S. Pat. No. 7,559,922, both of which are commonly assigned with the present application and are incorporated herein by reference.

In the compacted state 14, the lower rounded portion 24 may be inverted and fitted within the upper rounded portion 22 as best shown in FIG. 4. As such, the walls 30, 32 of the lower rounded portion 24 may be positioned between the inlet opening 40 and the filter 20 (which is arranged on an inner surface of the outer wall 32), such that the lower rounded portion 24 acts as a barrier to at least partially shield the filter 20 from contact with liquid and solid stomal discharge to extend filter life and enhance filter performance that may otherwise be degraded by partial or total blockage of the filter by stomal discharge. Although the filter 20 is illustrated as attached to the inner surface of the outer wall 32, in other embodiments, the filter may be attached on an outer surface of the pouch, or the pouch may not include a filter.

In use, a user can decide whether to wear the expandable ostomy pouch 10 in the compacted state 14 or in the expanded state 12. For example, at home, the user may want to wear the expandable pouch 10 in its expanded state 12, and may want to reduce the size of the pouch 10 to go out or to merely minimize the visual appearance of the pouch. When a smaller pouch is desired, the user can simply push in the lower rounded portion 24 with lingers, such that the lower rounded portion is inverted and fitted within the upper rounded portion. The expandable pouch 10 in its compacted state 14 is small and can provide added discretion in public, yet includes additional waste collection capacity in the contained lower rounded portion. Thus, sudden outflows of stomal discharge can be accommodated. As the stomal discharge fills the cavity 36, the lower rounded portion 24 expands due to gravity and/or due to elevated pressure within the pouch 10 due to the stomal discharge. Alternatively, the user can choose to expand the pouch 10 by simply pulling out the lower rounded portion 24.

In this embodiment, the lower rounded portion 24 is similarly sized as compared to the upper rounded portion 22, and thus provides for a greater waste collection capacity than the upper rounded portion 22. This is because the upper rounded portion 22 includes an inlet opening 40 to receive the stoma, which reduces the waste collection capacity of the upper rounded portion 22 to that volume of the pouch below the stoma. Therefore, it is contemplated that the expandable pouch 10 can provide at least a 100% increase in the waste collection capacity through the lower rounded portion 24, preferably at least a 120% increase in the waste collection capacity.

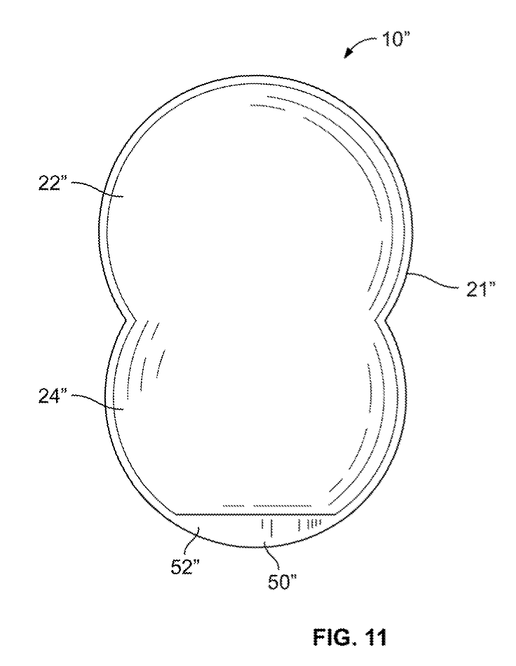

In some embodiments, the expandable ostomy pouch can include a tab that a user can grasp and pull out the tucked in lower portion to expand the pouch from a compacted state. FIG. 11 is an illustration of an expandable ostomy pouch embodiment including a tab. An expandable ostomy pouch 10″ is configured similar to the expandable ostomy pouch 10 of FIGS. 1-4, and has a “peanut” shape body 21″ including an upper rounded portion 22″ and a lower rounded portion 24″. The lower rounded portion 24″ includes a tab 50″ which can facilitate expansion of the ostomy pouch from a compacted state to an expanded state. In this embodiment, the tab 50″ is defined by a heat sealed portion 52″ in the lower part of the lower rounded portion 24″. Alternatively, the tab can be a separate piece attached to the lower rounded portion by heat sealing, adhesive or other suitable methods.

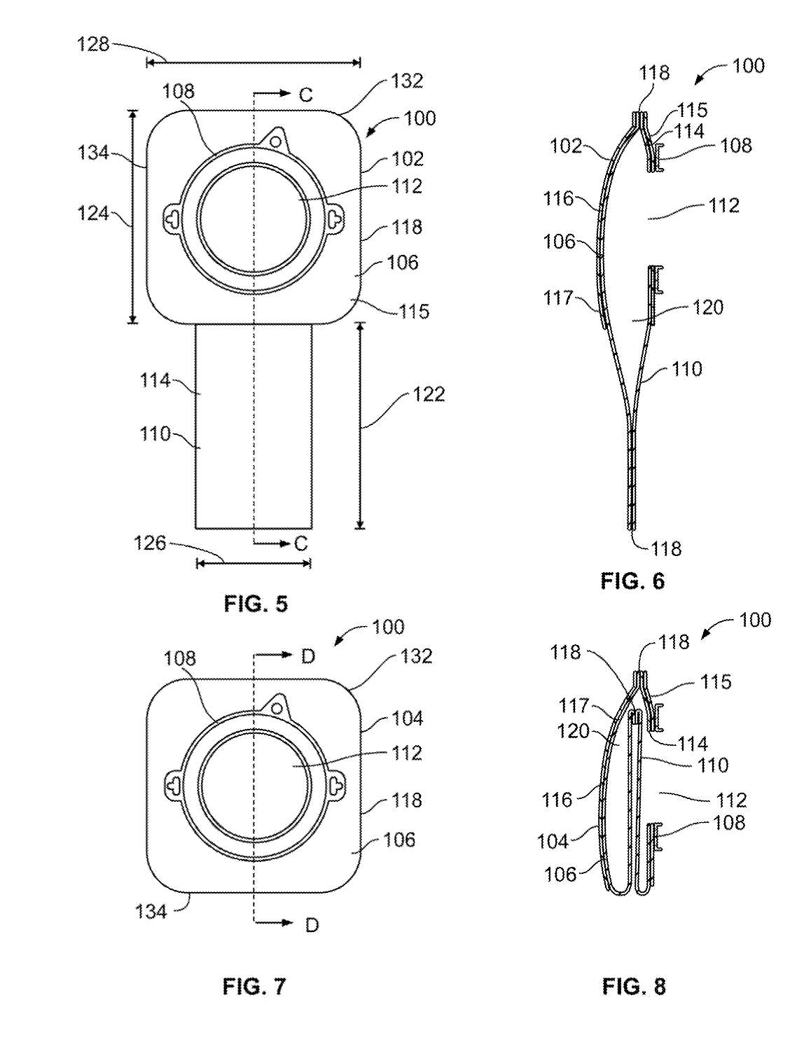

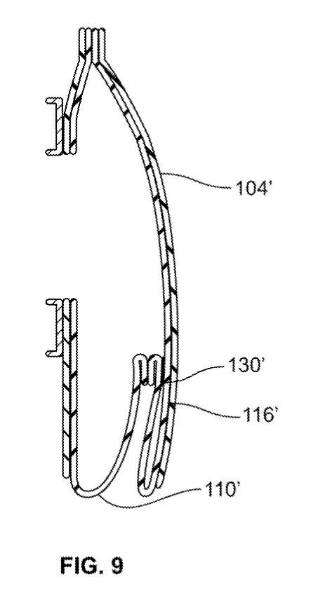

FIGS. 5-8 illustrate an expandable ostomy cap 100 according to another embodiment. FIGS. 5 and 6 show the expandable ostomy cap 100 in an expanded state 102, and FIGS. 7 and 8 show the expandable ostomy cap 100 in a compacted state 104. The expandable ostomy cap 100 includes, generally, a cap portion 106, a connecting flange 108 for securing the ostomy cap 100 to an adhesive barrier (not shown) placed around the stoma, and a tail pouch portion 110. Although expandable ostomy cap 100 is shown with a connecting flange, the expandable ostomy cap 100 can be provided with an adhesive skin barrier in other embodiments.

The expandable ostomy cap 100 may be formed of a bodyside wall 114 and an outer wall 116, which may be sealed along their peripheral edges 118 to define a cavity 120. The expandable ostomy cap depicted in FIGS. 5-8 is provided with non-woven layers 115, 117 on outer surfaces of the bodyside wall 114 and the outer wall 116 respectively. The non-woven layers 115, 117 are optional, and thus, may only be provided on one of the walls or may not be provided in other embodiments.

The cap portion 106 is similarly sized as known ostomy caps. For example, the cover portion 106 with a 50 mm diameter circular inlet opening 112 can have a rounded-edge square body 132 with about 4 inches length sides 134. In another embodiment, the cover portion can have a circular body having a diameter of about 4 inches.

To accommodate sudden outflows of stomal discharge, the expandable ostomy cap may be provided with the tail pouch portion 110. The tail pouch portion 110 may be configured to be inverted and inserted into the cap portion 106. Thus, the tail pouch portion 110 can be generally sized to be about same as the cap portion or smaller. In the embodiment shown, the expandable ostomy cap 100 is configured such that a length 122 of the tail pouch portion 110 and a length 124 of the cap portion 106 are about equal, and a width 126 of the tail pouch portion 110 is less than a width 128 of the cap portion 106. In other embodiments, the tail portion may have a length shorter than that of the cap portion, and a width equal to the width of the cap portion.

In the compacted state 104, the tail pouch portion 110 may be inverted and inserted into the cavity 120 of the cap portion 106, as shown in FIG. 8. As such, the expandable ostomy cap 100 in the compacted state has a size approximately equal to that of the cap portion 106, which is a typical ostomy cap size. Thus, the cavity 120 of the cap portion 106 is expected to have a relatively small collection capacity. However, with the hidden tail pouch portion 110, the collection capacity of the ostomy cap 100 is significantly increased. In one embodiment, the waste collection capacity of the ostomy cap 100 is increased by at least 50% in the expanded state, preferably by at least 100%, and more preferably by at least 120%.

To obtain the larger collection capacity, the user can simply pull out the tail pouch portion 110, in a similar manner to the expandable ostomy pouch 10. Alternatively, the user can allow the tail pouch portion 110 to expand due to gravity and/or due to elevated pressure within the cavity 120 due to the stomal discharge as the cavity 120 fills with stomal discharge.

In one embodiment, the tail pouch portion 110′ in the compacted state 104′ can be secured to an inner surface of the outer wall 116′ with a soluble film 130′. In such an embodiment, the expandable ostomy cap 100′ stays in the compacted state 104′ until the soluble film 130′ comes in contact with stomal discharge, at which time the soluble film 130′ is weaken and dissolved by fluid in stomal discharge, thereby releasing the tail pouch portion 110′ to expand. The soluble film 130′ provides added security in that the tail pouch portion 110′ is securely attached to the inner surface of the cap portion 106′ during exercise or movement until the expanded collection capacity is needed to accommodate stomal discharge.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. An expandable ostomy pouch, comprising:
a first portion;
a second portion, wherein the first portion and the second portion are defined by a bodyside wall and an outer wall, the bodyside will and the outer wall being sealed alone their peripheral edges to define a cavity, and the cavity being continuous through the first portion and the second portion; and
an inlet opening arranged on the bodyside wall of the first portion for receiving a stoma,
wherein the second portion is configured to be inverted and inserted within the first portion in a compacted state such that the second portion is substantially completely hidden within the first portion, and the second portion is substantially completely exposed from the first portion in an expanded state.

2. The expandable ostomy pouch of claim 1, wherein the expandable ostomy pouch is configured to expand from the compacted state to the expanded state by manually pulling the second portion out of the first portion, or due to gravity and/or due to elevated pressure within the expandable ostomy pouch due to the stomal discharge as the cavity is filled with stomal discharge.

3. The expandable ostomy pouch of claim 1, wherein the first portion and the second portion are configured such that the second portion is fitted, securely within the first portion, and does not expand out of the first portion during exercise or other user movement without manual expansion or expansion by stomal discharge.

4. The expandable ostomy pouch of claim 1, wherein the expandable ostomy pouch has a peanut-like shape, wherein the first portion has a substantially circular shape and the second portion has a substantially circular shape, the second portion being smaller in size than the first portion such that the second portion is inverted and securely fitted within the first portion

5. The expandable ostomy pouch of claim 1, wherein the first portion has a substantially square shape and the second portion has a substantially rectangular shape, wherein a width of the second portion is smaller than a width of the first portion, and a length of the second portion is smaller than a length of the first portion, wherein the expandable ostomy pouch has substantially the same shape and size as the first portion when in a compacted state.

6. The expandable ostomy pouch of claim 1, wherein the expandable ostomy pouch is a drainable pouch including an outlet opening defined at an end of the second portion and a closing means for closing the outlet opening, wherein the outlet opening is closed prior to being inverted and inserted within the portion in the compacted state.

7. The expandable ostomy pouch of claim 1, wherein the expandable ostomy pouch is a single use pouch free of an outlet opening.

8. The expandable ostomy pouch of claim 1, wherein a waste collection capacity of the expandable ostomy pouch in the expanded state is at least 50% greater than that in the compacted state.

9. The expandable ostomy pouch of claim 8, wherein a waste collection capacity of the expandable ostomy pouch in the expanded state is at least 100% greater than that in the compacted state.

10. The expandable ostomy pouch of claim 1, further including a non-woven material arranged on an outer surface of at least one of the bodyside wall and the outer wall, wherein the non-woven material extends along the first portion only.

11. The expandable ostomy pouch of claim 1, wherein the second portion includes a tab for facilitating expansion of the pouch from the compacted state to the expanded state, wherein the tab is configured such that a user can grasp the tab and pull out the second portion from within the first portion.

12. The expandable ostomy pouch of claim 11, wherein the tab is defined by a heat sealed portion in a lower part of the second portion.

13. The expandable ostomy pouch of claim 1, wherein the second portion is attached to the first portion on an inner surface of the outer wall with a water-soluble material.

14. The expandable ostomy pouch of claim 1, further including a filter arranged on an inner surface of the outer wall of the first portion, wherein the inverted second portion is arranged between the inlet opening and the filter in the compacted state to at least partially shield the filter from stomal discharge.

15. An expandable ostomy cap, comprising:
a cap portion, the cap portion sufficiently sized to cover a stoma and to support a means for attaching the expandable ostomy cap to a user;
a tail portion, wherein the cap portion and the tail portion are defined by a bodyside wall and an outer wall, the bodyside wall and the outer wall being sealed along their peripheral edges to define a cavity, and the cavity being continuous through the cap portion and the tail portion; and
an inlet opening arranged on the bodyside wall of the cap portion for receiving a stoma:
wherein the tail portion is configured to be inverted and inserted within the cap portion in a compacted state such that the tail portion is substantially completely hidden within the cap portion, and the second portion is substantially completely exposed from the first portion in an expanded state.

16. The expandable ostomy cap of claim 15, wherein the cap portion has a substantially square shape, wherein each side of the cap portion is between about 3.5 inches and about 5 inches.

17. The expandable ostomy cap of claim 15, wherein the cap portion has a substantially circular shape, wherein a diameter of the cap portion is between about 3.3 inches and about 5 inches.

18. The expandable ostomy cap of claim 15, wherein the cap portion and the tail portion are configured such that the tail portion is fitted securely within the cap portion and does not expand out of the cap portion during exercise or other user movement without manual expansion or expansion by stomal discharge.

19. The expandable ostomy cap of claim 15, wherein a waste collection capacity of the expandable ostomy cap in the expanded state is at least 100% greater than that in the compacted state.

20. The expandable ostomy cap of claim 15, wherein the tail portion is attached to the cap portion on an inner surface of the outer wall with a water-soluble material.
